(19) United States
(12) Patent Application Publication
(10) Pub. No.: US 2018/0092595 A1
(43) Pub. Date: Apr. 5, 2018

(54) SYSTEM AND METHOD FOR TRAINING AND MONITORING ADMINISTRATION OF INHALER MEDICATION

(71) Applicant: Mundipharma Laboratories Gmbh, Basel (CH)

(72) Inventors: Christopher Karl Chen, Singapore (SG); Loh Siew Leng, Singapore (SG); Ghulam Murtaza Khan Qasuri, Singapore (SG)

(21) Appl. No.: 15/712,217

(22) Filed: Sep. 22, 2017

Related U.S. Application Data
(60) Provisional application No. 62/403,777, filed on Oct. 4, 2016.

(51) Int. Cl. A61B 5/00; A61M 15/00; A61B 7/00; A61B 5/11

(52) U.S. Cl. CPC A61B 5/4833; A61B 5/0077; A61B 5/486; A61B 5/7425; A61M 15/00; G06K 9/00228; A61B 5/1123; A61B 5/1128; A61B 5/7475; A61B 5/6898; G06T 2207/30201; A61B 7/003

(57) ABSTRACT

Systems and methods are provided for training and monitoring administration of an inhaler medication. The system includes a mobile computing device that is configured to provide an augmented reality training and monitoring aid for asthma patients. In particular, the mobile device is programmed to capture video using a camera and sound recordings using a microphone in order to measure the patient's head position from the video and measure events relating to inhalation and exhalation from the microphone recordings. This real-time data is used to provide real-time testing and monitoring of the patient's technique for using an inhaler and an augmented reality training aid that informs the patients training. In addition, the mobile device is configured to collect background information from the patient relating to the patient's control over his or her asthma and can also interface with a back-end computing system for storing and maintaining related information.

### CROSS-REFERENCE TO RELATED APPLICATION

The present application claims priority to U.S. Provisional Patent Application No. 62/403,777, entitled SYSTEM AND METHOD FOR TRAINING AND MONITORING ADMINISTRATION OF INHALER MEDICATION, filed on Oct. 4, 2016, the contents of which are hereby incorporated by reference as if set forth in its entirety herein.

### BACKGROUND OF THE INVENTION

Asthma is a prevalent medical affliction shared by patients worldwide. A number of factors contribute to the effective treatment and control over asthma ranging from mental attitude toward the use of medication and the effectiveness of their dosage. For instance, a patient's attitude towards their asthma can play a significant role in the patient's adherence to asthma medication regimen, engagement with healthcare advice and the patient's overall level of control. Studies suggest that asthma patients respond differently to treatment based on their attitude toward asthma and patients generally have a poor perception of their level of asthma control. It has also been found that patients with problems often share common profiles/attributes. Unfortunately only a minority of the patients with asthma actually achieve good asthma control.

Incorrect inhaler technique is a significant concern when treating asthma. In particular, studies suggests that patients who are not in control of their asthma commonly lack control because they not using inhaler correctly rather than an incorrect medication or dosage.

Moreover, there are a few critical steps in the administration process can make a significant difference in the efficacy of the medication.

Existing methods for treating asthma are deficient for a number of reasons. Often incorrect inhaler usage stems from the fact that there are various types of inhalers such as Dry Powder Inhalers (“DPI”) and pressurized metered dose inhalers (“pMDI”), each requiring a particular technique for effective administration of the medication. In addition, poor technique also arises from poor patient training and limited capabilities of healthcare professionals, particularly in certain geographic areas. With respect to training, information that is provided through existing avenues, i.e., video, is common, but is hard to absorb and as such does not effectively instruct a patient. Moreover, it is challenging to assess patient usage of his or her inhaler and their control over asthma after the patient has left the controlled clinic setting and is using medication during daily life.

Accordingly, what is needed is a system that is capable of providing mindset-specific support, education and engagement with patients. Moreover, what is needed is a tool to guide patients on appropriate inhalation technique using augmented reality. Furthermore, what is needed is a training and monitoring tool that enables sharing of key control measures directly with healthcare professionals, if desired. Further what is needed is a system that can centrally aggregate de-identified patient data across all relevant metrics, to enable central review and interpretation and also utilize real-world earnings from across a population of patients to inform the treatment of other patients.

It is with respect to these and other considerations that the disclosure made herein is presented.

### BRIEF DESCRIPTION OF THE DRAWING FIGURES

FIG. 1 is a high-level diagram illustrating an exemplary system for training and monitoring administration of an inhaler medication according to an embodiment of the present invention;

FIG. 2 is a block diagram illustrating an exemplary configuration of a mobile device according to an embodiment of the present invention;

FIG. 3A is a flow diagram illustrating an exemplary method for profiling a patient according to an embodiment of the present invention;

FIG. 3B is a flow diagram illustrating an exemplary method for assessing a patient's control over asthma according to an embodiment of the present invention;

FIG. 3C is a flow diagram illustrating exemplary steps for advising a patient according to an embodiment of the present invention;

FIG. 4 is a flow diagram illustrating an exemplary method for training and testing a patient technique for administering medication using an inhaler according to an embodiment of the present invention;

FIG. 5 is a flow diagram illustrating an exemplary method for evaluating a patient technique for administering medication using an inhaler using video imagery according to an embodiment of the present invention; and

FIG. 6 is a flow diagram illustrating an exemplary method for evaluating a patient technique for administering medication using an inhaler based on audio data according to an embodiment of the present invention.

### DETAILED DESCRIPTION OF CERTAIN EMBODIMENTS AND OF ASPECTS OF THE INVENTION

By way of overview and introduction, what is provided is a system for training and monitoring administration of an inhaler medication 100. The system includes a mobile computing device that is specifically configured to provide an augmented reality training aide and monitoring device for asthma patients. According to a salient aspect, the mobile device implements a patient application that is programmed to utilize the mobile device camera and microphone to provide real-time testing and monitoring of the patient's technique for using an inhaler, and uses this real-time data to provide an augmented reality training aid that informs the patients training. In addition, the patient application is configured to collect background information from the patient relating to the patient's control over his or her asthma and also interface with a back-end computing system for storing and maintaining the patient's profile. It can be appreciated that patient-specific data can be stored on the mobile device. Information stored on the back-end computing system may be anonymized in one or more ways before it is stored or used, so that personally identifiable information is removed. For example, identifiers associated with a patient's identity, medical records and medical data collected using the app and the like may be anonymized so that no personally identifiable information can be determined for the patients from the de-identified data on the back-end system. This allows the patient to, at his or her option, provide health-care professionals with more comprehensive access to his or her information and more closely and effectively monitor the patient's control over his or her asthma. Accordingly, the system for training and monitoring administration of an inhaler medication is a holistic solution that provides an asthma coach and facilitates the ongoing sharing of information between the patient and doctor.

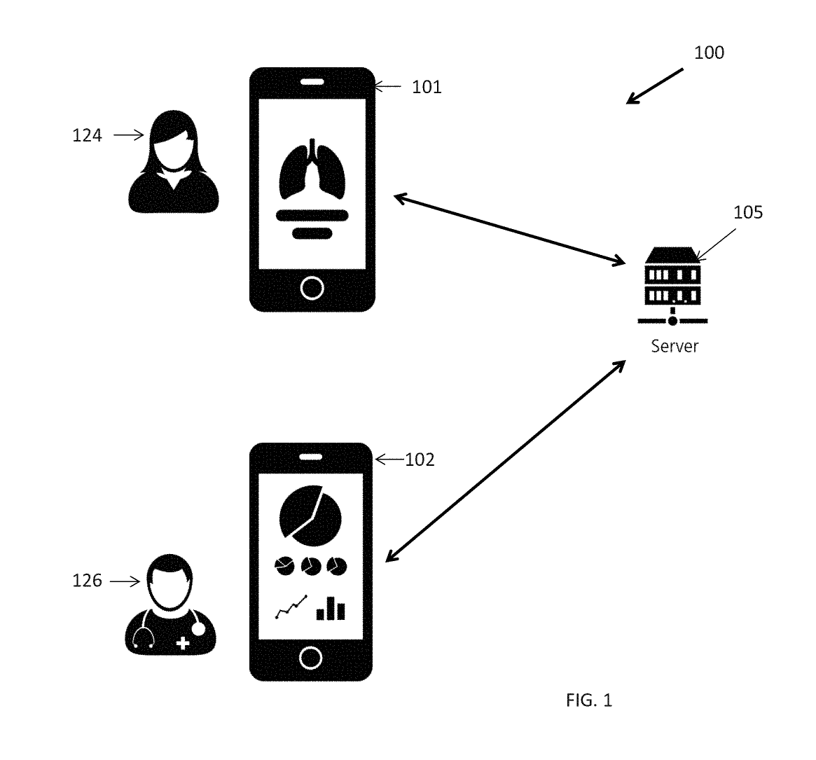

An exemplary system for training and monitoring administration of an inhaler medication 100 is shown as a block diagram in FIG. 1. In one arrangement, the system consists of a back-end system server 105 and user-side devices including a mobile device 101 and a personal computing device 102. As shown in FIG. 1, a patient 124 can use the mobile device 101 that is further configured to implement a patient application and can be in further communication with the back-end system server 105 via a network (not shown). Generally, the main aspects of the patient application include a patient profiling tool, a longitudinal control tool and an instruction and testing component. The instruction and testing component provide an augmented reality instructional tool and active monitoring of the patient during testing exercises and during the actual administration of medication. Also in communication with the back end system 105 is the computing device 102, which as shown can be used by a health-care professional 126 to access the patient's information stored on the back-end server. As noted above, any information that is provided to or provided to and stored by the back-end system or accessed via the back-end system can be maintained in a de-identified format. Thus, it should be apparent that in the exemplary system and routines described herein, a patient can opt in, thereby consenting to the storage of the de-identified patient information by the back-end system as well as any other information that he or she provides and consents to use by the system.

As further described herein, the system 100, facilitates training and monitoring administration of an inhaler medication using, among other things, video imagery and sound data captured by a patient using the mobile device 101. In accordance with the disclosed embodiments, the mobile device 101 is used to train the patient as to the proper procedure for administering medication using an inhaler and can also be used to monitor the patient's use of the inhaler to administer medication. As shown, the computing device 102 can be used by healthcare professionals to access and review records associated with the patient's training and ongoing use of the inhaler as recorded with the mobile device 101. The access to information by a health-care professional can be contingent upon patients providing express consent for such access. Similarly, the patient can provide the health-care professional the information stored on their device via email or other direct electronic transmission. In some implementations, the de-identified information could be accessed by the healthcare professional indirectly via the back-end system server 105. It can be further appreciated that the patient can also access stored information on the system server 105 or otherwise interact with the back-end system using his or her personal computing device like computing device 102, which is further described herein as being used by the healthcare professional.

The system server 105 can be practically any computing device and/or data processing apparatus capable of communicating with the user devices and receiving, transmitting and storing electronic information and processing requests as further described herein. The user devices, i.e., mobile device 101 and personal computing device 102, can be configured to communicate with the system server 105, transmitting electronic information thereto and receiving electronic information therefrom as further described herein. The user-side devices can also be configured to receive user inputs as well as capture and process biometric information, for example, digital images and sound recordings of a patient 124.

The mobile device 101 can be any mobile computing devices and/or data processing apparatus capable of embodying the systems and/or methods described herein, including, but not limited to, a personal computer, tablet computer, personal digital assistant, mobile electronic device, a wearable electronic device, a cellular telephone, or a smart phone device. The computing device 102 is intended to represent various forms of personal computing devices that the healthcare professional can interact with, such as a personal computer, laptop computer, smartphone or other appropriate personal digital computers.

It should be noted that while FIG. 1 depicts the system for training and monitoring administration of an inhaler medication 100 with respect to a mobile device 101 and a computing device 102, any number of such devices can interact with the system in the manner described herein. It should also be noted that while FIG. 1 depicts a system for training and monitoring administration of an inhaler medication 100 with respect to the patient 124 and healthcare professional 126, any number of such users can interact with the system in the manner described herein.

It should be further understood that while the various computing devices and machines referenced herein, including but not limited to mobile device 101 and system server 105 and personal computing device 102, are referred to herein as individual/single devices and/or machines, in certain implementations the referenced devices and machines, and their associated and/or accompanying operations, features, and/or functionalities can be combined or arranged or otherwise employed across any number of such devices and/or machines, such as over a network connection or wired connection, as is known to those of skill in the art.

It should also be understood that the exemplary systems and methods described herein in the context of the mobile device 101 (also referred to as a smartphone) are not specifically limited to the mobile device and can be implemented using other enabled computing devices (e.g., the personal computing device 102).

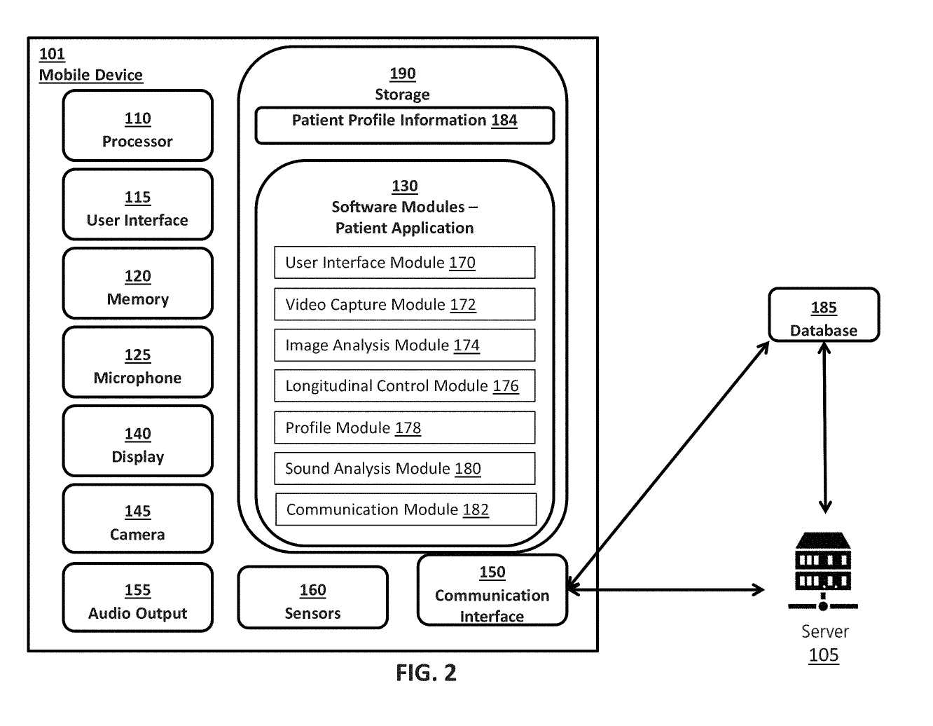

In reference to FIG. 2, the mobile device 101 includes various hardware and software components that serve to enable operation of the system, including one or more processors 110, a memory 120, a microphone 125, a display 140, a camera 145, an audio output 155, a storage 190 and a communication interface 150. Processor 110 serves to execute or otherwise implement a patient application in the form of software instructions that can be loaded into memory 120. Processor 110 can be a number of processors, a central processing unit CPU, a graphics processing unit GPU, a multi-processor core, or any other type of processor, depending on the particular implementation.

Preferably, the memory 120 and/or the storage 190 are accessible by the processor 110 and comprise one or more non-transitory storage media, thereby enabling the processor to receive and execute instructions encoded in the memory and/or on the storage so as to cause the mobile device and its various hardware components to carry out operations for aspects of the systems and methods as will be described in greater detail below. Memory can be, for example, a random access memory (RAM) or any other suitable volatile or non-volatile computer readable storage medium. In addition, the memory can be fixed or removable. The storage 190 can take various forms, depending on the particular implementation. For example, the storage can contain one or more components or devices such as a hard drive, a flash memory or some combination of the above. Storage also can be fixed or removable.

One or more software modules 130 are encoded in the storage 190 and/or in the memory 120. The software modules 130 can comprise one or more software programs or applications having computer program code or a set of instructions (also referred to as the “patient application”) executed in the processor 110. As depicted in FIG. 2, preferably, included among the software modules 130 is a user interface module 170, a video capture module 172, an image analysis module 174, a longitudinal control module 176, a profile module 178, a sound analysis module 180 and a communication module 182 that are executed by processor 110. Such computer program code or instructions configure the processor 110 to carry out operations of the systems and methods disclosed herein and can be written in any combination of one or more programming languages. Preferably, the program code executes entirely on mobile device 101, as a stand-alone software package. However, in some implementations, the program code can also execute partly on mobile device and partly on system server 105, or entirely on system server or another remote device. In the latter scenario, the remote systems can be connected to mobile device 101 through any type of network (not shown), including a local area network (LAN) or a wide area network (WAN), mobile communications network, cellular network, or the connection can be made to an external computer (for example, through the Internet using an Internet Service Provider).

It can also be said that the program code of software modules 130 and one or more computer readable storage devices (such as memory 120 and/or storage 190) form a computer program product that can be manufactured and/or distributed in accordance with the present invention, as is known to those of ordinary skill in the art. It should also be understood that in some illustrative embodiments, one or more of the software modules 130 can be downloaded over a network to storage 190 from another device or system via communication interface 150.

As will be described in greater detail below, the storage 190 preferably contains and/or maintains various data items and elements that are utilized throughout the various operations of the system and method for training and monitoring administration of an inhaler medication 100. The information stored in storage can include but is not limited to a patient profile 184, which includes information relating to: the patient's asthma condition, the patient's medication, the patient's performance of training exercises and testing, the patient's control over his or her asthma, overall health and the like, as will be described in greater detail herein. It should be noted that although storage is depicted as being configured locally to mobile device 101, in certain implementations the storage and/or the data elements described as being stored therein can also be located remotely, such as on a remote database 185 that is accessible to the system server 105, and can be accessible to the user-side devices through a network in a manner known to those of ordinary skill in the art.

A user interface 115 is also operatively connected to the processor. The interface can be one or more input or output device(s) such as switch(es), button(s), key(s), a touch-screen, microphone, etc. as would be understood in the art of mobile devices. User interface serves to facilitate the capture of commands from the user (e.g., on-off commands) or patient information and settings related to operation of the system 100. For example, interface serves to facilitate the capture of certain information from the mobile device 101 such as personal patient information for enrolling with the system so as to create a patient profile.

The computing device 101 can also include a display 140 which is also operatively connected to processor the processor 110. The display includes a screen or any other such presentation device which enables the system to instruct or otherwise provide feedback to the user regarding the operation of the system for 100. By way of example, the display can be a digital display such as a dot matrix display or other 2-dimensional display. By way of further example, the interface and the display can be integrated into a touch screen display. Accordingly, the display is also used to show a graphical user interface, which can display various data, provide interactive “forms” that allow for the entry of information by the patient, virtual buttons and the like. Touching the touch screen at locations corresponding to the display of a graphical user interface allows the person to interact with the device to enter data, change settings, control functions, etc.

Mobile device 101 also includes a camera 145 capable of capturing digital images. The camera can be one or more imaging devices configured to capture images of at least a portion of the patient's body including the patient's head and/or face while utilizing the mobile device 101. The camera serves to facilitate the capture of images of the patient for the purpose of image analysis by the mobile device processor 110 executing the patient application. Image analysis functions include identifying the patient's head and face and evaluating the patient during training stages and during use of the inhaler. The mobile device 101 and/or the camera 145 can also include one or more light emitters (e.g., LEDs, not shown) for example, a visible light emitter/flash. Preferably, the camera is a front-facing camera that is integrated into the mobile device and incorporates an optical sensor, for example and without limitation a CCD or CMOS sensor. As would be understood by those in the art, camera 145 can also include additional hardware such as lenses, light meters and other conventional hardware and software features that are useable to adjust image capture settings such as zoom, focus, aperture, exposure, shutter speed and the like. Alternatively, the camera can be a rear facing camera or external to the mobile device 101 and connected electronically to the processor 110. The possible variations of the camera would be understood by those skilled in the art.

In addition, the mobile device can also include one or more microphones 125 for capturing audio recordings. The hardware and associated software applications for recording sound using a microphone would be understood by those skilled in the art. In addition, in some implementations, the microphone can be an external microphone that is communicatively connected to the processor, for instance, a microphone that is connected to the mobile device via a headphone jack or other hard-wired or wireless data connection.

Audio output 155 is also operatively connected to the processor 110. Audio output can be any type of speaker system that is configured to play audio data files as would be understood by those skilled in the art.

Various hardware devices/sensors 160 can also be operatively connected to the processor. The sensors 160 can include: an on-board clock to track time of day and otherwise time events, as further described herein; an accelerometer to track the orientation and acceleration of the mobile device; Gravity magnetometer to determine the 3-dimensional orientation of the mobile device; proximity sensors to detect a distance between the mobile device and other objects such as the patient and other such devices as would be understood by those skilled in the art.

While certain of the components utilized in the monitoring system and method are understood devices, their coordination under program control and the combination of particular resources (such as on-board camera, clock, processor, GPS, and so on) to implement the monitoring system and method provides technological advances in the art of unsupervised administration of inhaled medications by patients.

At various points during the operation of the system for training and monitoring administration of an inhaler medication 100, the mobile device 101 can communicate with one or more computing devices, such as system server 105. Such computing devices transmit and/or receive data to/from mobile device 101, thereby preferably initiating maintaining, and/or enhancing the operation of the system 100, as will be described in greater detail below. Accordingly, a communication interface 150 is also operatively connected to the processor 110 and can be any interface that enables communication between the mobile device 101 and external devices, machines and/or elements including system server 105. Preferably, communication interface includes, but is not limited to, a modem, a Network Interface Card (NIC), an integrated network interface, a radio frequency transmitter/receiver (e.g., Bluetooth, cellular, NFC), a satellite communication transmitter/receiver, an infrared port, a USB connection, and/or any other such interfaces for connecting the mobile device to other computing devices and/or communication networks such as private networks and the Internet. Such connections can include a wired connection or a wireless connection (e.g. using the 802.11 standard) though it should be understood that communication interface can be practically any interface that enables communication to/from the mobile device.

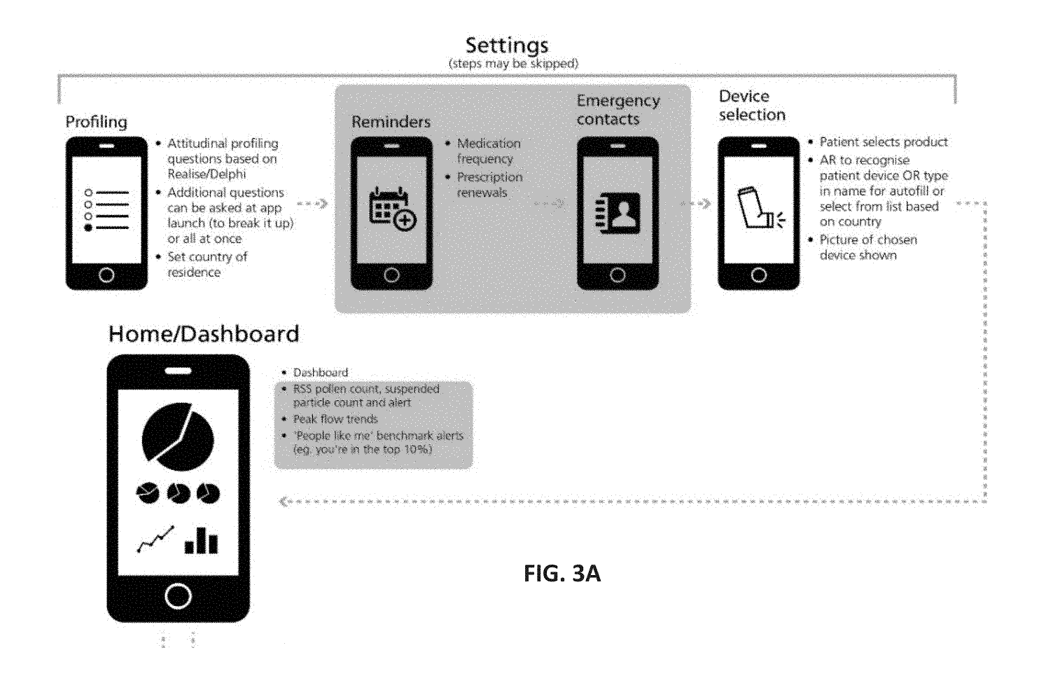

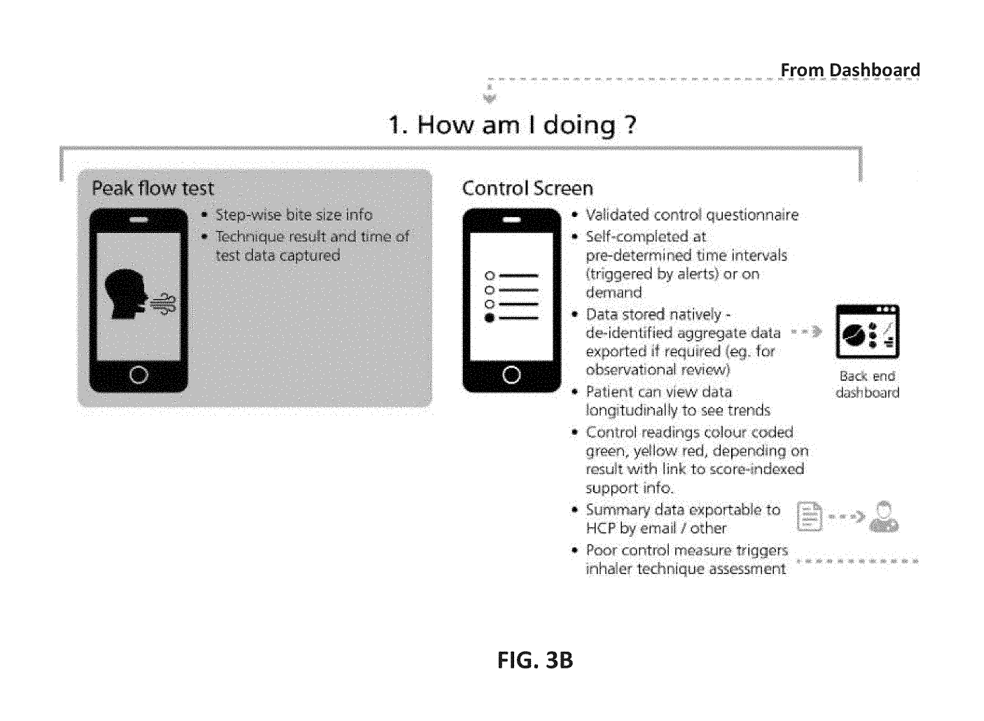

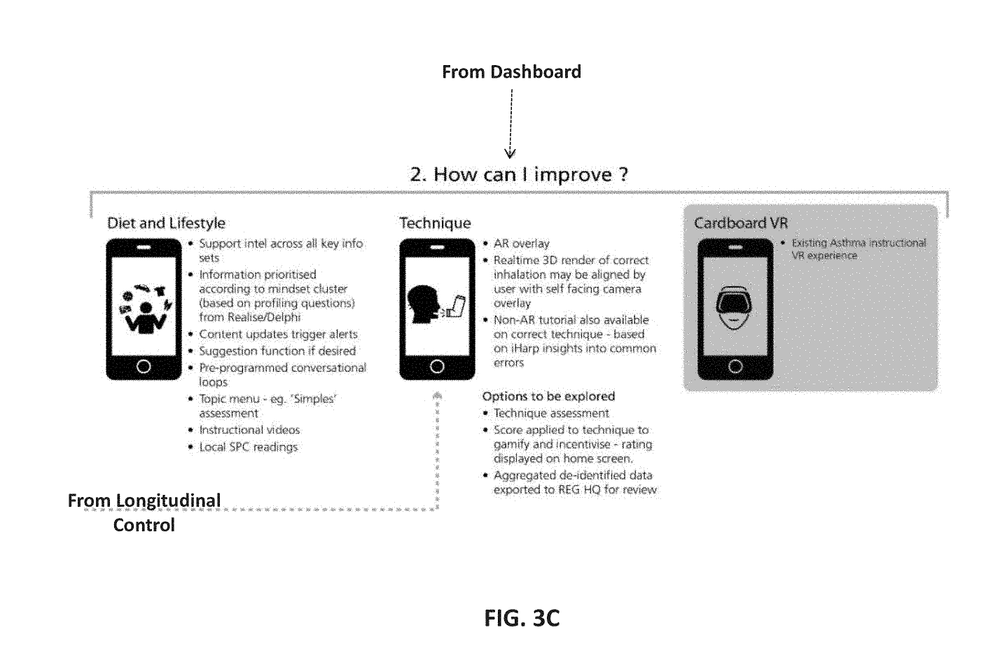

FIG. 3A, includes a high-level overview and flow-chart including steps directed to the registration of a patient and developing a patient profile. As noted above, one important component of effective monitoring and treatment of a patient is developing a patient profile. The patient profiling tool implemented by the mobile device processor executing the patient application, in particular, the patient profile module performs steps for gathering information from the patient for the purposes of registering the patient, gathering baseline information relating to the patient's condition, and defining the settings for the patient. As shown in FIG. 3A, the steps include: administering a questionnaire to develop an attitudinal profile for the patient. For instance the patient profile can be generated using questions according to their feelings and attitudes toward having asthma. Additional patient profile and registration information can also be collected, for instance the patient's country of residence, prescription information and medication frequency and the like. In addition, patient registration can also include identification of the particular type of inhaler device that is used by the patient (e.g., a DPI or pMDI inhaler). In some implementations the patient can select the particular inhaler manually via the mobile device user-interface 115. In addition or alternatively, the processor 110, which is configured by executing one or more of the software modules 130 including the video capture module 172 and image analysis module 174 and profile module 178, can prompt the patient to capture images of the patient's inhaler using the camera 145 and can analyze the imagery to identify the particular type of inhaler.

As shown in FIG. 3A, once a patient has completed the initial profiling and registration sequence, the mobile device processor executing the patient application can be configured to present the patient with a “dashboard.” The dashboard user interface preferably presents the patient with information that is relevant to the patient's medical condition. For instance, the configured processor present real-time environmental information collected from data sources such as pollen count, air pollution, temperature, and other location specific environmental circumstances. The dashboard can also collect and present metrics based on the patient's personal data and data provided by the system server 105 relating to other patients in the area, for instance, peak flow trends in area. Accordingly, the dashboard can not only advise the patient as to their personal progress but also provide a benchmark for the patient based on similar patients. In addition, the mobile device processor can also be configured to assist with the patient's medication regimen by providing alerts and reminders through the dashboard. Moreover, the dashboard can also provide the patient with access to the remaining evaluation and testing tools provided by the patient application.

As noted above, another important component of the patient application and ongoing monitoring of the patient's condition is the longitudinal control tool. FIG. 3B includes a high-level overview and process flow diagram illustrating the mobile device 101 and various stages in the process for monitoring a patient's longitudinal control. In particular, the processor 110, which is configured by executing one or more of the software modules 130 including the longitudinal control module 176, can evaluate the patient's longitudinal control by administering to the patient a validated control questionnaire via the mobile device display 140. The configured processor can also be configured to analyze the patient's answers to the questionnaire and objectively measure whether patient has his or her asthma under control and how the asthma is being controlled. For instance, the control questionnaire can be administered to determine whether the patient is using his or her inhaler as a preventative measure or as a rescue tool. It should also be appreciated that the particular patient's attitudinal profile as described in relationship to FIG. 3A dictates the frequency of their interaction with the application.

The longitudinal control testing steps can also include prompting patients to take measurements relating to their medical condition. In some implementations, the patient can be prompted to take a peak-flow test using an electronic peak-flow meter that is in data communication with the processor 110 such that the processor can record and analyze the data captured from the peak-flow meter. For instance, the peak-flow meter can be plugged into the headphone jack of the mobile device or in wireless communication with the mobile device using a wireless communication connection (e.g., Bluetooth or WWI connection).

In addition, one or more steps of the longitudinal control process can be repeated periodically after the initial registration. For instance the questionnaire can be administered at pre-determined time intervals, based on the occurrence of certain events (e.g., decrease in asthma control). Accordingly, the configured processor is able to monitor changes in the patients control over the asthma and objectively evaluate the impact of the patient's treatment using the patient application. It can be appreciated that steps of the profiling process can similarly be repeated.

It should be appreciated that data collected using the patient profiling and longitudinal control tools, as well as the information collected during training and patient monitoring further described herein, can be stored natively on the mobile device, for instance in storage 190. In addition, the data can be exported to the system server 105 for storage in the database 185. Accordingly, de-identified data gathered from multiple patients can be presented to patients or healthcare professionals, for instance, in order to benchmark a patient's condition against other patients as mentioned above. The system server 105 can also be configured to provide summaries of such collected data to the patients electronically via email or other communication systems. These summaries can include grades/scores generated using the mobile device and/or the system server 105 based on the collected data.

Based on the patient's longitudinal control, patients can be presented with additional information and guidance to help the patient improve their control over their condition. For instance, as shown in FIG. 3C, the patient can be provided with additional information relating to the patient's lifestyle, diet, and overall health. Moreover, depending on the patient's longitudinal control, as measured using the configured processor 110, the patient can also be prompted to undergo further training and evaluation of the patient's technique for administering medication using an inhaler device, as further described herein. It should be appreciated that the guidance information, training and evaluation tools that are provided by the patient application to improve the patient's control can also be initiated by the patient manually (e.g., from the dashboard or other such home-screen of the patient application) or automatically.

With respect to the instruction and testing tools, the mobile device processor 110 executing one or more of the software modules 130 of the patient application is configured to capture real-time video of the patient using the camera 145, display the real time video to the patient on the display 140 and also overlay/render additional digital content on the screen so as to provide an augmented reality tutorial to the patient. In addition, the processor is also configured to analyze the real-time video and audio data captured using a microphone 125 and evaluate/grade the patient's technique for administering medication using an inhaler and dynamically update and modify the instruction and augmented reality experience accordingly.

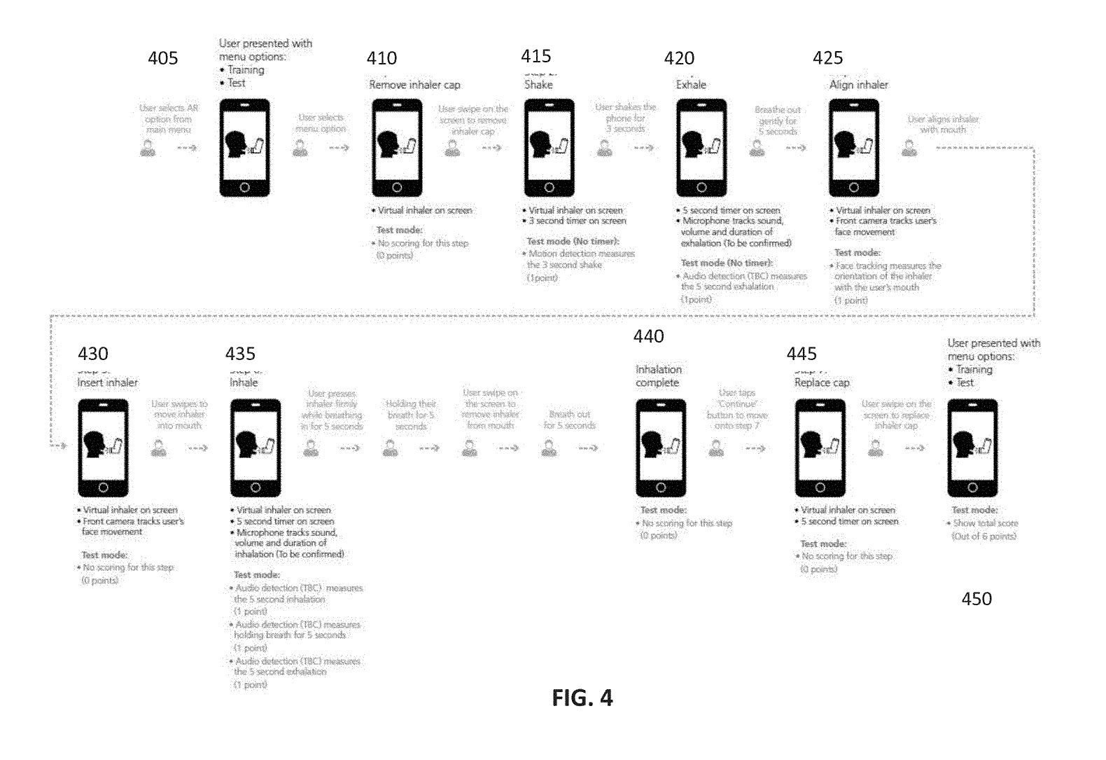

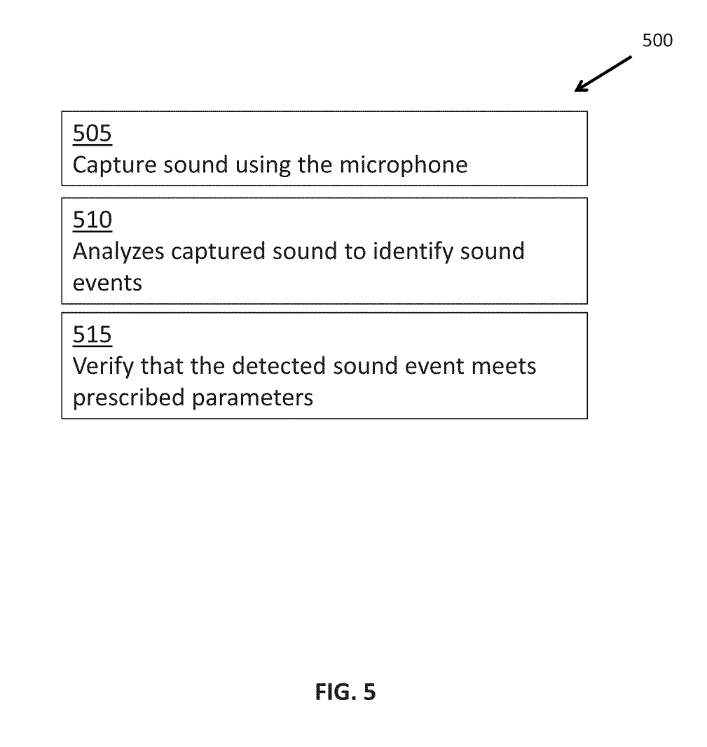

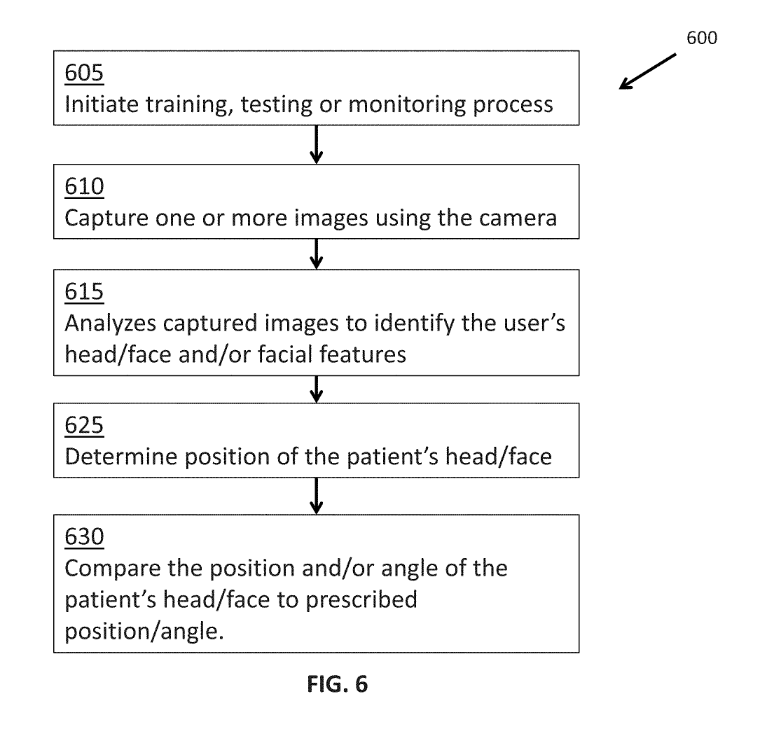

An exemplary process for training and testing a patient's technique for administering medication using an inhaler is shown in FIG. 4. The steps described herein, and in regard to each of the flow diagrams, are implemented by a processor under control of executable code/instructions stored in the memory or storage of the device. The code is configured to direct the resources available to the device to capture images, obtain data, communicate with remote devices, and so on. A more specific discussion of the steps for monitoring the patient's technique from sound data and video imagery are further described below in relation to FIGS. 5 and 6, respectively.

As shown in FIG. 4, the process begins at step 405, where the patient is presented with a menu of options including training and testing. Both training and testing processes provide an augmented reality experience on the mobile device and incorporate the specific processes for monitoring the patient's technique using imagery captured using the camera and sound data collected using the microphone. Training includes steps for instructing the patient on the various steps and requirements for proper and effective use of an inhaler. Testing is provided to evaluate whether the patient's technique is adequate for administering the medication using an inhaler. Although the particular combination of steps that are further described herein are directed to a pMDI inhaler, it can be appreciated that the augmented-reality tutorial and the steps described during training/testing, as well as the particular technique that is evaluated using real-time video and sound data can be tailored to any number of different inhalers (e.g., DPI or pMDI).

Upon receiving the patient's selection of the training or testing option, at step 410, the patient is presented with a virtualized inhaler depicted on the screen. In training mode, the patient can be prompted to remove the cap of the virtualized inhaler by swiping the screen. Then at step 415, the patient can be prompted to shake the “virtual” inhaler for a prescribed amount of time, say, three seconds. In training mode, a three second timer can be displayed on the screen prompting the patient to shake the phone for three seconds to simulate shaking of the inhaler. During both training and testing, the processor 110 can be configured to verify that the prompted event occurred, namely, determine from the accelerometer data whether the patient shook the phone for three seconds. Then at step 420, the patient can be prompted to exhale. In training mode, another timer can be displayed on the screen prompting the patient to exhale for a prescribed about of time, say, five seconds. In both training and testing modes, the processor 110 can be configured to verify that the prompted event occurred. For instance, as described below in relation to FIG. 5, the processor can use the microphone to capture sound and verify from the captured sound data whether the volume and duration of the exhalation event meets the prescribed requirements. Based on the analysis of the sound data, the processor can grade the exhalation and issue a score or pass/fail for the particular step. In training mode, if the patient fails the particular test, the patient can be prompted to repeat the step and can also be provided with additional instruction and information.

Then at step 425, the patient can be prompted to align the inhaler with his or her mouth. In particular, the processor 110 can display a virtual inhaler on the screen/display 140 superimposed over the real-time video of the patient's face captured using a camera 145 on the device. In some implementations, the camera can be a “front facing camera” (e.g., exposed on the same side of the device as the display) such that the patient can be imaged while the patient is viewing the display of the camera 140. In some implementations the rear facing camera (e.g., a camera exposed on the opposite side of the display) can be used, for instance, in cases where a doctor, parent or other person is filming the patient while the patient is performing the training or testing steps.

In both training and testing modes, the processor 110 can be configured to verify that the prompted event occurred. For instance, as described below in relation to FIG. 6, the processor can analyze the video imagery to verify that the patient's mouth is aligned with the mouth of the inhaler and the patient's head is in alignment with the inhaler. Based on the analysis of the image data, the processor can grade the patient's head position and issue a score or pass/fail grade for the particular step. In training mode, if the patient fails the particular test, the patient can be prompted to repeat the step and can also be provided with additional instruction and information.

Then at step 430, the patient can be prompted to move the inhaler into the patient's mouth. For instance, during training mode, the patient can be prompted to swipe the screen indicating the patient completed this step.

Then at step 435, the patient can be prompted to actuate the inhaler and then perform the inhalation, hold and exhalation steps. For instance, in training mode, another timer can be displayed on the screen and the patient can be prompted to actuate the virtual inhaler (e.g., push a button on the mobile device) and inhale for a prescribed about of time, say, five seconds. In both training and testing modes, the processor 110 can be configured to verify that the prompted events occurred. For instance, as described below in relation to FIG. 5, the processor can use the microphone to capture sound and verify from the captured sound data that the patient has begun to inhale. Accordingly, the processor can start the timer displayed on the screen. In addition, the processor can also analyze the sound data to determine whether the volume and duration of the inhalation event meets the prescribed requirements. Moreover, the processor can determine whether the inhalation step was followed by a five second period in which the patient was holding his or her breath. In other words, detect the absence of an inhalation or exhalation event for five seconds. Subsequently, the processor can also measure whether the breath-hold period was followed by a five second exhalation. Meanwhile during these individual stages, the processor can be adjusting the feedback provided on the screen, including without limitation, the instructions for the particular step and the associated timer. Based on the analysis of the sound data, the processor can grade the inhalation, hold and exhalation steps and a grade for the individual steps and entire process. In training mode, if the patient fails the test for one or more of the stages, the patient can be prompted to repeat the step and can also be provided with additional instruction and information.

Thereafter, at steps 440-445, the patient can be prompted to continue the training or testing process and then replace the cap of the virtualized inhaler. In addition, at step 450, the patient can be provided with an overall score of the patient's technique and presented with menu options to repeat the training or testing procedure. In regard to scoring, the configured processor tests a number of different components of the inhaler administration process (e.g., head position, inhaler alignment, inhalation, exhalation and the like) scores each stage and can determine pass/fail for individual components as well as the overall process. In addition, as noted above, the results of the instruction and testing procedure can be recorded into the patient's profile locally on the mobile device and/or remotely onto the system server 105. Accordingly both the patient and a healthcare professional can review and evaluate the patient's record. Similarly, the information gathered during active monitoring of inhaler use (i.e., after training and testing) can be recorded into the patient's profile in a similar fashion.

An exemplary process for evaluating the patient's technique based on sound information captured using the microphone is further described herein in relation to FIG. 5. The process 500 begins at step 505, when the mobile device processor, which is configured by executing one or more of the software modules 130 including, without limitation, the sound analysis module 180, captures the ambient sound using the microphone 125. Preferably, the sound is captured during one or more steps of the medication administration process (e.g., exhalation, inhalation of training process 400) and records the sound data to the device memory 120 or storage 190.

Then, at step 510, the configured processor 110 analyzes the captured sound recording to identify and classify events. For example, the sound detection algorithm can be specifically trained to detect sounds associated with breathing (i.e., inhalation and exhalation). Similarly, the sound detection algorithm can also be trained to detect events associated with a patient's use of the inhaler such as the shaking of the inhaler, depressing/actuating the inhaler and the like. In particular, the sound detection algorithm can be specifically trained based on sound clips captured using the microphone while the patient is performing various actions during a set-up process to identify the characteristic sound of the various events performed by the during inhaler use. In addition or alternatively, the sound detection algorithm can be trained based on sound data captured from a plurality of different patients. Moreover, the sound detection algorithm can be trained to detect and classify breathing events based on the distinct sounds associated with breathing events having certain characteristics. For instance, the particular sound characteristics of a breathing event can indicate the volume of air inhaled or exhaled as well as the force of the inhalation and exhalation. In addition, the sound analysis module 180 can also configure the processor 110 to determine the length of the inhalation or exhalation event based on the length of the detected sound. More specifically, the length of an event can be determined by detecting the start of the event and monitoring the elapsed time, as determined from an on-board clock, until the particular sound ceases to be detected by the processor.

In some implementations, the processor 110 executing the user interface module 170 can also be configured to prompt the patient to interact with the device before performing a particular training or administration step. For instance, the patient can be asked to push a virtual button or physical button before performing the step of inhaling for five seconds. Accordingly, based on the received user input, the processor 110 can activate the appropriate sensor device (e.g., microphone, camera, accelerometer) and/or start a timer during which the sensor is recording and the processor is analyzing the recorded data to detect the corresponding event.

Similarly, it can be appreciated that the configured processor can prompt the patient to perform various user-input actions in order to simulate a particular action relating to administration of an inhaler medication. For instance, the patient can be prompted to push a physical button on the mobile device in order to simulate pressing/actuating the inhaler. Thereafter the mobile device can be configured to record audio data and analyze the data to determine whether the patient inhaled for a prescribed amount of time and with the prescribed volume and/or force of inhalation.

Thereafter, at step 515, the configured processor can compare the detected sound and associated characteristics to a prescribed set of parameters that are associated with proper execution of the particular step of the medication administration process. For instance, the processor can determine whether the captured inhalation event lasted the prescribed duration and was indicative of an inhaled breath having at least the prescribed volume. Based on the comparison, the processor 110 can also generate a score for patient's performance of the particular step.

FIG. 6 depicts an exemplary method 600 for evaluating the position of the patient's head while performing one or more of the steps for administering medication using an inhaler. Such image-based grading of the patient's physical technique for administering the inhaler medication can be implemented by the processor 110 of the mobile device 101, which is configured by executing instructions in the form of one or more of the software modules 130 including, preferably, the video capture module 172 and the image analysis module 174, and using the camera 145 of the mobile device 101. The process is initiated at step 605. In some implementations the image capture and image analysis process can be initiated automatically by the processor or in response to a patient interacting with one or more buttons on the mobile device, for example, a button provided on the smartphone or a virtual button provided by a touchscreen display.

At step 610, the configured processor causes the camera to capture one or more images, preferably, of at least a portion of the patient's head including the face and can receive the images from the camera for further analysis. In addition, at step 610, the processor can display the captured images back to the patient via the display 140. Accordingly, the patient can be provided with feedback in the form of the captured images in near-real time during the testing and monitoring process.

In addition, at step 610, the configured processor can render a guide on the display 140. In some implementations the guide can include one or more vertical or horizontal lines that are superimposed over the real-time video. The guide can prompt the patient to hold the mobile phone and camera in a particular orientation and can also prompt the patient to position the patient's head relative to the camera in an ideal manner. It can be appreciated that additional shapes can be used as a guide, for instance, an oval can be rendered over the real-time video stream so as to simulate the shape of a patient's head and also prompt the patient to fill the oval space with the patient's face thereby causing the patient to position the patient's face at an appropriate distance from the camera.

In some implementations, particularly during the training process, rendering the guide can include superimposing a virtualized inhaler onto the screen. Accordingly, the patient can, based on the position of the inhaler relative to the real-time image of the patient's face, align the inhaler with the patient's mouth. Otherwise, during monitoring while using actual inhaler, the guide can be provided so as to prompt the patient to position his or her head at an appropriate distance or angle relative to the camera.

At step 615, the configured processor analyzes one or more of the captured images to identify the patient's face and/or one or more facial features of the patient's face. For instance, the processor can implement a face detection algorithm or a feature detection algorithm, as would be understood by those in the art, to detect the patient's face or facial features. In some implementations, the facial features that are identified can include one or more of: the mouth, eyes, nose, chin, forehead, neck, cheeks and the like.

Then at step 625, the configured processor can determine the angle of the patient's head relative to the camera. In some implementations, the absolute location (i.e., planar coordinates) of the face and/or the detected facial features within one or more of the captured images can be determined. In addition or alternatively, a relative location of facial features can be determined. For instance, the position of the eyes relative to one-another or the patient's mouth can be determined and used to verify whether the head is vertically aligned with the camera. It can also be appreciated that verifying the alignment of the patient's head in the side-to-side direction can be determined as a function of the angle of the camera being held by the patient. Accordingly the angle of the patient's head can be determined irrespective of whether the patient is holding the camera at an angle. By way of further example, the vertical alignment of the patient's nose and mouth in the one or more images can indicate that the patient's head is vertically aligned with the camera.

In some instances, it is also preferable for patients to tilt their head back when administering medication using an inhaler. Accordingly, at step 625, the processor can also be configured to determine the angle of the patient's head in a front-to-back direction. In some implementations, this can include determining a distance of certain facial features from the camera based on the captured images and comparing the distance to determine the angle of the patient's head. By way of example and without limitation, determining the relative distance of facial features can include capturing imagery of the patient's face while sweeping the focal distance of the lens and then analyzing the sharpness of various facial features depicted in the captured images to determine the distance of the feature from the camera based on the corresponding focal distance for the analyzed images. In addition, the angle of the head can be determined based on the shape of the captured head and/or face. For instance, a template of the shape of the patient's face or head when tilted back (or a relative position of specific facial features) can be determined during a set-up process. Accordingly, during subsequent patient training and monitoring processes, the processor can determine the angle of the patients head by comparing the shape of the patient's face, as determined from a current set of images, to the prescribed template to verify that head angle in the captured image(s) is consistent with the template. Alternative processes for determining distance of the patient's facial features from the camera images can also be implemented, as would be understood by those in the art.

It can also be appreciated that, in addition or alternative to determining position of the head based on a position of the features relative to one-another, or relative to the camera, the position and angle of the patient's head, face or facial features can be determined based on the position of the features of interest relative to the guides that have been superimposed onto the captured images, for instance the virtualized inhaler. Accordingly, in some implementations, the position of the patient's mouth relative to the position of the virtualized inhaler can be determined and graded so as to verify that the patient knows to position his or her mouth on the inhaler mouthpiece.

Then at step 630, the processor 110 can determine whether the patient has positioned his or her head or face as instructed. For instance, when administering medication with an inhaler, the patient's head is preferably held in line with the patient's neck, in other words, strait in a vertical direction. Accordingly, based on the orientation of the mobile device when the images are captured, as determined from an on-board accelerometer, and the position of the patient's facial features determined at step 625, the processor can determine whether the patient's head is vertically aligned.

In addition, at step 630, the processor can also determine whether the angle of the patient's head in a front-to-back direction is appropriate for effectively administering medication using an inhaler. For instance, based on a comparison of the depth of certain facial features, say, the forehead and the mouth, the processor can determine if the patient has angled his or her head back to a prescribed degree. Based on the comparison of the measured position of the head (e.g., in one or more of the side-to-side or front-to-back direction) to the prescribed parameters, the processor can generate a score for the patients head-position.

The subject matter described above is provided by way of illustration only and should not be construed as limiting. Various modifications and changes can be made to the subject matter described herein without following the example embodiments and applications illustrated and described, and without departing from the true spirit and scope of the present invention, which is set forth in the following claims.

It is to be understood that like numerals in the drawings represent like elements through the several figures, and that not all components and/or steps described and illustrated with reference to the figures are required for all embodiments or arrangements.

The flowchart and block diagrams in the figures illustrate the architecture, functionality, and operation of possible implementations of systems, methods and computer program products according to various embodiments and arrangements. In this regard, each block in the flowchart or block diagrams can represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

What is claimed is:

1. A method for monitoring asthma control by a patient using an inhaler device based on real-time sensor data captured using a mobile computing device, comprising:
administering, with the mobile device, an inhaler alignment test including:
capturing, by the mobile device having a camera, a non-transitory storage medium, instructions stored on the storage medium, and a processor configured by executing the instructions, a sequence of images depicting a face of the patient;
detecting, with the processor, at least a portion of a head of the patient;
superimposing, with the processor in the sequence of images, a virtualized inhaler device;
displaying to the patient using a display of the mobile device, the sequence of images including the superimposed inhaler;
determining, with the processor using the sequence of images, a position of the head relative to one or more of the camera and the virtualized inhaler
measuring, with the processor using the sequence of images, an angle of the patient's head based on the determined position of the head relative to one or more of the camera and the virtualized inhaler;
administering, with the mobile device, one or more breathing event tests including:
prompting the patient to perform one or more breathing events including one or more of an inhalation of air and an exhalation of air;
capturing, with the processor using a microphone, audio-data of the one or more breathing events;
determining, with the processor from the audio-data using a sound analysis algorithm, a duration of the one or more breathing events and an estimated volume of air inhaled or exhaled during the one or more the breathing events;
testing, with the processor, the patient's performance of the one or more breathing events by:
comparing the determined duration and volume of the one or more breathing events to prescribed parameters associated with the one or more breathing events;
testing, with the processor, the patient's performance of the inhaler alignment test by:
comparing the measured angle of the patient's head to a prescribed angle; and
generating, with the processor, a score for the patient's performance based on a result of one or more of the testing steps.

2. The method of claim 1, wherein:
the step of administering the one or more breathing event tests comprises performing as a first breathing event, an exhalation of air, and performing as a second breathing event, an inhalation of air, and
wherein the step of generating the score is performed for each of the first and second breathing events.

3. The method of claim 1, further comprising:
re-administering one or more of the inhaler alignment test and the one or more breathing event tests in response to the score generated for a respective test being below a prescribed level.

4. The method of claim 1, wherein the step of testing the patient's performance of the inhaler alignment test further comprises:
verifying, with the processor based on the determined position of the head relative to the virtualized inhaler, that the patient's mouth is aligned with a mouth of the inhaler.

5. The method of claim 1 further comprising:
outputting a prompt, by the processor using the display, that instructs the patient to perform an act using the mobile device;
detecting, by the processor, a user interaction with the mobile device in response to the prompt; and
verifying, by the processor based on the detected user interaction and prescribed parameters for the act, that the user performed the act using the device in accordance with the prescribed parameters associated with the act.

6. The method of claim 5, wherein the act comprises an interaction with the user interface simulating removing a cap of the virtualized inhaler displayed on the display, and wherein the detected user interaction is a gesture performed by the user and received by the processor via the user interface.

7. The method of claim 5, wherein the act comprises an interaction with the mobile device including shaking the mobile device for a prescribed period of time;
wherein the detecting step comprises measuring, by the processor using an accelerometer in data communication with the processor, movement of the mobile device; and
wherein the verifying step comprises determining, by the processor based on the measured movement, that the movement corresponds to a user shaking the mobile device for a prescribed period of time.

8. The method of claim 1, further comprising:
administering, by the mobile device, a longitudinal control test comprising:
displaying, with the processor using the display, a longitudinal control questionnaire that prompts the patient to input answers to the questionnaire via the user interface, and
measuring, by the processor based on the patient's answers received via the user interface, the patient's level of control over their asthma condition and how the patient is controlling his or her asthma condition; and
performing the steps of administering the inhaler alignment test and the one or more breathing event tests based on the measured level of control.

9. The method of claim 8, wherein administering the longitudinal control test further comprises:
prompting the patient to perform a peak-flow test using an electronic peak-flow meter that is in data communication with the processor;
capturing, by the processor using the peak-flow meter, peak-flow data for the patient; and
measuring, by the processor, the patient's asthma condition based on the captured peak-flow data.

10. The method of claim 9, further comprising:
periodically re-administering one or more steps of the longitudinal control test over a period of time; and
monitoring, with the processor, changes in the patient's level of control over the period of time.

11. A method for providing a patient with a system for monitoring asthma control by the patient using an inhaler device based on real-time sensor data received at a mobile computing device of the type having a camera, a non-transitory storage medium, instructions stored on the storage medium, a microphone, a display and a processor configured by executing the instructions, comprising providing to the mobile device:
a software application which comprises one or more software modules that configure the processor to administer an inhaler alignment test, including:
a video capture module that, when executed by the processor, configures the processor to capture, using the camera, a sequence of images depicting a face of the patient;
an image analysis module that configures the processor to:
detect at least a portion of a head of the patient in the sequence of images,
superimpose a virtualized inhaler device in the sequence of images,
display the sequence of images including the superimposed virtualized inhaler to the patient via the display,
determine, using the sequence of images, a position of the head relative to one or more of the camera and the virtualized inhaler,
measure an angle of the patient's head based on the determined position of the head relative to one or more of the camera and the virtualized inhaler, and
generate a score for the patient's performance of the inhaler alignment test by comparing the measured angle of the patient's head to a prescribed angle;
wherein the software application further comprises one or more software modules that, when executed by the processor, configures the processor to administer one or more breathing event tests, including:
a sound analysis module that configures the processor to:
prompt the patient to perform one or more breathing events including one or more of an inhalation of air and an exhalation of air,
capture, using the microphone, audio-data of the one or more breathing events,
determine from the audio-data using a sound analysis algorithm, a duration of the one or more breathing events and an estimated volume of air inhaled or exhaled during the one or more the breathing events and
generate a score for the patient's performance of the one or more breathing event tests by comparing the determined duration and volume of the one or more breathing events to prescribed parameters associated with the one or more breathing events; and
wherein the software application further comprises a user interface module that configures the processor to generate an alert based on one or more of the generated scores for the patient's performance of the one or more breathing events and the inhaler alignment test, and output the alert to the user via the mobile device.

12. A system for monitoring asthma control by a patient using an inhaler device based on real-time sensor data received at
a mobile computing device of the type having a camera, a non-transitory storage medium, instructions stored on the storage medium, a microphone, a display and a processor configured by executing the instructions, comprising:
a software application comprising one or more software modules that configure the processor to administer an inhaler alignment test, including:
a video capture module that, when executed by the processor, configures the processor to capture, using the camera, a sequence of images depicting a face of the patient;
an image analysis module that configures the processor to:
detect at least a portion of a head of the patient in the sequence of images,
superimpose a virtualized inhaler device in the sequence of images,
display the sequence of images including the superimposed virtualized inhaler to the patient via the display,
determine, using the sequence of images, a position of the head relative to one or more of the camera and the virtualized inhaler,
measure an angle of the patient's head based on the determined position of the head relative to one or more of the camera and the virtualized inhaler, and
generate a score for the patient's performance of the inhaler alignment test by comparing the measured angle of the patient's head to a prescribed angle;
wherein the software application further comprises one or more software modules that, when executed by the processor, configures the processor to administer one or more breathing event tests, including:
a sound analysis module that configures the processor to:
prompt the patient to perform one or more breathing events including one or more of an inhalation of air and an exhalation of air,
capture, using the microphone, audio-data of the one or more breathing events,
determine from the audio-data using a sound analysis algorithm, a duration of the one or more breathing events and an estimated volume of air inhaled or exhaled during the one or more the breathing events and
generate a score for the patient's performance of the one or more breathing event tests by comparing the determined duration and volume of the one or more breathing events to prescribed parameters associated with the one or more breathing events; and
wherein the software application further comprises a user interface module that configures the processor to generate an alert based on one or more of the generated scores for the patient's performance of the one or more breathing events and the inhaler alignment test, and output the alert to the user via the mobile device.

13. The system of claim 12, wherein the processor is configured to:
execute a test of a first breathing event among the one or more breathing events performed by the user, wherein the first breathing event comprises an exhalation of air, and generate a first breathing event score for the first breathing event;
based on the first score exceeding a threshold score, administer the inhaler alignment test and generate an alignment score for the inhaler alignment test; and
based on the inhaler alignment test exceeding a threshold score, execute a test of a second breathing event among the one or more breathing events performed by the user, wherein the second breathing event comprises an inhalation of air, and generate a second breathing event score for the second breathing event;

14. The system of claim 12, wherein the image analysis module further configures the processor to verify, based on the determined position of the head relative to the virtualized inhaler, that the patient's mouth is aligned with a mouth of the inhaler and generate the score for the patient's performance of the inhaler alignment test as a function of the verification.

15. The system of claim 12, wherein the user interface module further configures the processor to output a prompt on the display instructing the patient to perform an act using the mobile device, detect a user interaction with the device in response to the prompt, and verify, based on the detected user interaction and prescribed parameters for the act, that the user performed the act using the device in accordance with the prescribed parameters associated with the act.

16. The system of claim 15, wherein the act comprises an interaction with the user interface simulating removing a cap of the virtualized inhaler displayed on the display, and wherein the detected user interaction is a gesture performed by the user and received by the processor via the user interface.

17. The system of claim 12, wherein the act comprises an interaction with the mobile device including shaking the mobile device for a prescribed period of time, and wherein the processor is configured to detect the user interaction by measuring movement of the mobile device using an accelerometer in data communication with the processor, and wherein the processor verifies that the user performed the act by determining that the measured movement of the mobile device corresponds to a user shaking the device for a prescribed period of time.

18. The system of claim 12, further comprising:
a longitudinal control module that, when executed by the processor, configures the processor to administer a longitudinal control test by:
displaying, using the display, a longitudinal control questionnaire that prompts the patient to input answers to the questionnaire via the user interface, and
measuring, based on the patient's answers received via the user interface, patient's level of control over their asthma condition and how the patient is controlling their asthma condition; and
wherein the processor is further configured to administer the inhaler alignment test and the one or more breathing event tests based on the measured level of control.

19. The system of claim 18, wherein the longitudinal control module further configures the processor to prompt the patient to perform a peak-flow test using an electronic peak-flow meter that is in data communication with the processor, capture peak-flow data for the patient using the peak-flow meter, and measure the patient's asthma condition based on the captured peak-flow data.

20. The system of claim 19, wherein the processor is configured to periodically re-administer one or more steps of the longitudinal control test over a period of time and monitor changes in the patient's level of control over the period of time.
